SEQUENCE LISTING (24 sequences)

SEQ ID NO: 1 - DNA, 24 bp, Artificial Sequence
  Description of Artificial Sequence Synthetic primer
acccttccttatcccttgatgcca

SEQ ID NO: 2 - DNA, 24 bp, Artificial Sequence
  Description of Artificial Sequence Synthetic primer
aaacagctctgcttcaacctgctg

SEQ ID NO: 3 - DNA, 24 bp, Artificial Sequence
  Description of Artificial Sequence Synthetic primer
agttcgacaagtgcatggtgtgtg

SEQ ID NO: 4 - DNA, 25 bp, Artificial Sequence
  Description of Artificial Sequence Synthetic primer
atggtgaccacgttgttgtatccgt

SEQ ID NO: 5 - DNA, 24 bp, Artificial Sequence
  Description of Artificial Sequence Synthetic primer
tcaccagcattgacgcatccaaac

SEQ ID NO: 6 - DNA, 24 bp, Artificial Sequence
  Description of Artificial Sequence Synthetic primer
ttcgtggtaggtccagcaaacagt

SEQ ID NO: 7 - DNA, 26 bp, Artificial Sequence
  Description of Artificial Sequence Synthetic primer
taaggatgcctggcccttccttcaaa

SEQ ID NO: 8 - DNA, 24 bp, Artificial Sequence
  Description of Artificial Sequence Synthetic primer
ggccactgacaagtttcaggcatt

SEQ ID NO: 9 - DNA, 24 bp, Artificial Sequence
  Description of Artificial Sequence Synthetic primer
accctggagcactcatgtttccta

SEQ ID NO: 10 - DNA, 24 bp, Artificial Sequence
  Description of Artificial Sequence Synthetic primer
tggcatcaagggataaggaagggt

SEQ ID NO: 11 - DNA, 24 bp, Artificial Sequence
  Description of Artificial Sequence Synthetic primer
catcctgttgttgctgtggctgat

SEQ ID NO: 12 - DNA, 24 bp, Artificial Sequence
  Description of Artificial Sequence Synthetic primer
gcgtggatgccgcatatgaagtta

SEQ ID NO: 13 - DNA, 24 bp, Artificial Sequence
  Description of Artificial Sequence Synthetic primer
agtttgaatgggcctttgagcacc

SEQ ID NO: 14 - DNA, 24 bp, Artificial Sequence
  Description of Artificial Sequence Synthetic primer
tagtccttgccagggaaagtcaca

SEQ ID NO: 15 - DNA, 22 bp, Artificial Sequence
  Description of Artificial Sequence Synthetic primer
atcacacgcccacagagatcca

SEQ ID NO: 16 - DNA, 22 bp, Artificial Sequence
  Description of Artificial Sequence Synthetic primer
gcttcaggctgttgagccatgt

SEQ ID NO: 17 - DNA, 20 bp, Artificial Sequence
  Description of Artificial Sequence Synthetic primer
gtaacccgttgaaccccatt

SEQ ID NO: 18 - DNA, 20 bp, Artificial Sequence
  Description of Artificial Sequence Synthetic primer
ccatccaatcggtagtagcg

SEQ ID NO: 19 - DNA, 23 bp, Artificial Sequence
  Description of Artificial Sequence Synthetic primer
atccattggagggcaagtctggt

SEQ ID NO: 20 - DNA, 26 bp, Artificial Sequence
  Description of Artificial Sequence Synthetic primer
gctcccaagatccaactacgagcttt

SEQ ID NO: 21 - protein, 24 aa, Artificial Sequence
  Description of Artificial Sequence Synthetic peptide
DRQIKIWFQNRRMKWKKRDVLPGT

SEQ ID NO: 22 - protein, 7 aa, Artificial Sequence
  Description of Artificial Sequence Synthetic peptide
RDVLPGT

SEQ ID NO: 23 - protein, 7 aa, Artificial Sequence
  Description of Artificial Sequence Synthetic peptide
RDVLPGT

SEQ ID NO: 24 - protein, 27 aa, Artificial Sequence
  Description of Artificial Sequence Synthetic peptide
RQIKIWFQNRRMKWKKRDVLPGTCVNS

(19) United States
(12) Patent Application Publication
(10) Pub. No.: US 2013/0297023 A1
(43) Pub. Date: Nov. 7, 2013

(54) Methods and Devices For Treating Intervertebral Disc Disease

(71) Applicants: Hee-Jeong Im Sampen, Lincolnwood, IL (US); Howard S. An, Glenview, IL (US); Di Chen, Chicago, IL (US)

(72) Inventors: Hee-Jeong Im Sampen, Lincolnwood, IL (US); Howard S. An, Glenview, IL (US); Di Chen, Chicago, IL (US)

(21) Appl. No.: 13/833,777

(22) Filed: Mar. 15, 2013

Related U.S. Application Data
(60) Provisional application No. 61/643,560, filed on May 7, 2012.

(51) Int. Cl. A61F 2/44

(52) U.S. Cl. CPC A61F 2/442
USPC 623/17.11

(57) ABSTRACT

Provided are methods for treating intervertebral disc degeneration, promoting tissue regeneration in the intervertebral disc, promoting homeostasis of the intervertebral disc, preventing intervertebral disc degeneration, and reducing inflammation of the intervertebral disc in a subject in need thereof comprising administering a myeloid differentiation primary response (88) (MyD88) antagonist to the subject. Also provided is a device, biomaterial or putty for implantation in or near the spine, wherein the device, biomaterial or putty is coated or infused with a myeloid differentiation primary response gene (88) (MyD88) antagonist.

### CROSS-REFERENCE TO RELATED APPLICATION

This application claims the benefit of U.S. Provisional Application No. 61/643,560, filed May 7, 2012, which is incorporated herein by reference in its entirety.

### TECHNICAL FIELD

The disclosure herein relates generally to methods and devices for providing anti-inflammatory and anti-catabolic effects, for example, in the treatment of intervertebral disc disease.

### BACKGROUND

The lifetime prevalence of back pain in the United States is 70-85% with roughly 10-20% of the population experiencing chronic symptoms (Andersson, 1999). While the etiology of back pain is multifactorial, it has been associated with degeneration of the intervertebral disc (IVD) (Mooney, 1989; Freemont et al., 2001). Disc degeneration results from an imbalance between degradation (catabolism) and synthesis (anabolism) of extracellular matrix (ECM) components by chondrocytes residing in the nucleus pulposus (NP) (Iatridis et al., 1997; Nomura et al., 2001). Through upregulation of molecules such as pro-inflammatory cytokines and catabolic growth factors, homeostasis of the ECM shifts toward a degenerative, catabolic state with subsequent breakdown of ECM components, including collagen fibrils surrounding and restraining large, hydrated aggregates of proteoglycans (PGs), principally aggrecan (Goldring, 2000; Masuda, 2006; Lee et al., 2009). For example, a relative increase in destructive enzymes, such as matrix metalloproteases (MMPs) or a disintegrin-like and metalloprotease with thrombospondin motifs (ADAMTS family, aggrecanases), may alter the normal balance within the disc and result in degeneration (Crean et al., 1997; Martel-Pelletier et al., 2001; Le Maitre et al., 2004; Im et al., 2007a; Im et al., 2007b; Le Maitre et al., 2007; Muddasani et al., 2007). Mammalian toll-like receptors (TLRs) comprise a family of at least 11 members that are involved in innate and adaptive immune responses, and specific members have been implicated in chronic inflammatory processes associated with cartilage degeneration (Takeda et al., 2003; Takeda and Akira, 2005; Kim et al., 2006; Liu-Bryan and Terkeltaub, 2010). All TLRs are type 1 transmembrane proteins that recognize distinct ligands extracellularly and mediate binding to adaptor proteins intracellularly. Myeloid differentiation primary response gene (88) (MyD88) is an adapter protein that links TLRs and Interleukin-1 receptors (IL-1Rs) with downstream signaling molecules. In cartilage, TLR-2 and TLR-4 are highly upregulated in joints with advanced osteoarthritis (OA) (Kim et al., 2006). Similarly, marked upregulation of TLR-2 and TLR-4 gene expression is observed in degenerative human spine components, including discs, facet joints and facet joint capsules, compared to those in age-matched normal tissues (unpublished data). Both receptors are sensitive to binding by bacterial lipopolysaccharides (LPS), which are microbial constituents found in the outer membrane of gram-negative bacteria. LPS has been implicated in both inflammatory and degenerative states in articular cartilage and bovine IVD tissue (Jasin, 1983; Aota et al., 2006), and the literature suggests that LPS induces catabolic effects via TLR-2 and 4, with destructive activity mediated mainly via LPS-induced activation of both receptor complexes in articular chondrocytes (Kim et al., 2006; Bobacz et al., 2007).

Despite several studies identifying TLR activity in articular cartilage and arthritic joints, however, there have been no reports in the literature on the functional activity of TLRs in spine tissue.

Although treatments for back pain exist, there remains a need for additional treatments, especially ones that correct the underlying pathological conditions that lead to back pain.

### SUMMARY

Disclosed herein are methods, devices, biomaterials and putties for treating intervertebral disc disease. Certain embodiments provide methods for treating intervertebral disc degeneration in a subject in need thereof comprising administering a MyD88 antagonist to the subject. Also provided are methods for promoting tissue regeneration in the intervertebral disc in a subject in need thereof comprising administering a MyD88 antagonist to the subject. Also provided are methods for promoting homeostasis of the intervertebral disc in a subject in need thereof comprising administering a MyD88 antagonist to the subject. Also provided are methods for preventing intervertebral disc degeneration in a subject in need thereof comprising administering a MyD88 antagonist to the subject. Further provided are methods for reducing inflammation of the intervertebral disc in a subject in need thereof comprising administering a MyD88 antagonist to the subject.

In other embodiments are provided a device, biomaterial or putty for implantation in or near the spine, wherein the device, biomaterial or putty is coated with a MyD88 antagonist.

### BRIEF DESCRIPTION OF THE DRAWINGS

FIG. 1 shows that inhibition of the MyD88 pathway counteracts LPS- and IL-1-induced catabolic effects in bovine NP cells. The monolayer of bovine NP cells were treated with MyD88i (MyD88 pathway specific peptide inhibitor, 100 μM), LPS (10 μg/mL), IL-1 (10 ng/mL) and combinations of either LPS and MyD88i or IL-1 and MyD88i. After stimulation for 24 hr, cells were harvested and total RNA was extracted to perform real time qPCR targeting key cartilage degrading enzymes including MMP-13 (A), ADAMTS-4 (B), ADAMTS-5 (C) and the MyD88 pathway receptor, TLR-2 (D). Values are the mean and SD and are representative of 3 different donors in 3 separate experiments. A value of p<0.05 and p<0.01 indicate a significant and a highly significant difference in ANOVA, respectively.

FIG. 2 shows that inhibition of the MyD88 pathway suppresses LPS- and IL-1-induced expression of MMP-13 in human NP cells. The monolayer of human NP cells were treated with MyD88i (100 μM), LPS (10 μg/mL), IL-1 (10 ng/mL) and combinations of either LPS and MyD88i or IL-1 and MyD88i for 24 hr. After stimulation, cells were harvested and subjected to either total RNA extraction for real time qPCR (A) or preparation of cell lysates for western blotting (B) to assess the altered level of a potent collagen-degrading enzyme MMP-13 using human specific anti-MMP-13 antibody. Values are the mean and SD and are representative of 3 different donors in 3 separate experiments. A value of p<0.05 and p<0.01 indicate a significant and a highly significant difference in ANOVA, respectively.

FIG. 3 shows that the LPS- and IL-1-induced expression of matrix-degrading enzyme MMP-1 is significantly inhibited by MyD88 peptide inhibitor in human NP cells. The monolayer of human NP cells were treated with MyD88i (100 μM), LPS (10 μg/mL) and IL-1 (10 ng/mL) and combinations of either LPS and MyD88i or IL-1 and MyD88i for 24 hr. (A) After stimulation, cells were harvested and total RNA was extracted to perform real time qPCR targeting MMP-1 gene expression. (B), Cell lysates were then prepared and analyzed by western blotting with human specific anti-MMP-1 antibody. Values are the mean and SD and are representative of 3 different donors in 3 separate experiments. A value of p <0.05 and p <0.01 indicate a significant and a highly significant difference in ANOVA, respectively.

FIG. 4 shows that the inhibition of the MyD88 pathway suppresses LPS- and IL-1-induced expression of aggrecanase (ADAMTS-4) and oxidative stress associated gene (iNOS) in human NP cells. Human NP cells in monolayer were treated with MyD88i (100 μM), LPS (10 μg/mL) or IL-1 (10 ng/mL) and combinations of either LPS and MyD88i or IL-1 and MyD88i for 24 hr. After stimulation, cells were harvested and total RNA was extracted to perform real time qPCR to assess aggrecanase ADAMTS-4 (A) and oxidative stress associated gene iNOS (B). A value of p<0.05 and p<0.01 indicate a significant and a highly significant difference in ANOVA, respectively.

FIG. 5 shows that the MyD88 inhibitory peptide antagonizes LPS- and IL-1-induced protease activity in human NP cells. Human NP cells in monolayer were treated with MyD88i (100 μM), LPS (10 μg/mL) or IL-1 (10 ng/mL) and combinations of either LPS and MyD88i or IL-1 and MyD88i for 24 hr. Gelatin zymography was performed by loading equal volumes of the conditioned media sample on polyacrylamide gel (A). Band images were digitally captured and the intensity of the bands (pixels/band) was obtained using the ImageJ densitometry analysis software in arbitrary optical density units (B). A value of p<0.01 indicates a highly significant difference in ANOVA.

FIG. 6 shows histological assessments (original magnification: X100). Lumbar spine discs of mouse were dissected after en bloc intradiscal pre-microinjection with MyD88i at the concentration of 150 μM per disc using 30 G needle (1.5 μL in volume). The MyD88i pre-injected discs were challenged with either IL-1 (100 ng/mL) or LPS (10 μg/mL) in DMEM/Ham's F-12 medium supplemented with 1% mini-ITS and further incubated for 6 days. Harvested discs were fixed, decalcified in EDTA and were paraffin embedded followed by serial disc sections of exactly 5-nm thickness to prepare slides. Safranin O-fast green staining was performed to assess general morphology and the loss of PG in disc ground substance.

FIG. 7 shows the chemical structure of peptidomimetic ST2825.

FIG. 8A shows the chemical structure of Compound 1 with different groups highlighted. FIG. 8B shows the chemical structure of Compound 1.

FIG. 9 shows the chemical structure of EM-163.

### DETAILED DESCRIPTION OF ILLUSTRATIVE EMBODIMENTS

The present subject matter may be understood more readily by reference to the following detailed description which forms a part of this disclosure. It is to be understood that the disclosed subject matter is not limited to the specific products, methods, conditions or parameters described and/or shown herein, and that the terminology used herein is for the purpose of describing particular embodiments by way of example only and is not intended to be limiting.

Each of the patents, patent applications, publications, and references cited herein is incorporated by reference in its entirety.

As employed above and throughout the disclosure, the following terms, unless otherwise indicated, shall be understood to have the following meanings.

As used herein, the singular forms “a,” “an,” and “the” include the plural reference unless the context clearly indicates otherwise. Further, unless otherwise required by context, singular terms shall include pluralities and plural terms shall include the singular.

Unless otherwise defined herein, scientific and technical terms used in connection with the present application shall have the meanings that are commonly understood by those of ordinary skill in the art.

As employed above and throughout the disclosure, the following terms and abbreviations, unless otherwise indicated, shall be understood to have the following meanings.

In the present disclosure, reference to a particular numerical value includes at least that particular value, unless the context clearly indicates otherwise. When a range of values is expressed, another embodiment includes from the one particular and/or to the other particular value. Similarly, when values are expressed as approximations, by use of the antecedent “about,” it is understood that the particular value forms another embodiment. All ranges are inclusive and combinable.

As used herein, the term “treatment” or “therapy” (as well as different forms thereof) includes preventative (e.g., prophylactic), curative or palliative treatment. As used herein, the term “treating” includes alleviating or reducing at least one adverse or negative effect or symptom of a condition, disease or disorder. This condition, disease or disorder can be intervertebral disc disease.

As employed above and throughout the disclosure the terms “effective amount” and “therapeutically effective amount” are used interchangeably to refer to an amount effective, at a dosage and for a period of time necessary, to achieve the desired result with respect to the treatment of the relevant disorder, condition, or side effect. It will be appreciated that the effective amount of components as disclosed herein will vary from patient to patient not only with the particular compound, component or composition selected, the route of administration, and the ability of the components to elicit a desired result in the individual, but also with factors such as the disease state or severity of the condition to be alleviated, hormone levels, age, sex, weight of the individual, the state of being of the patient, and the severity of the pathological condition being treated, concurrent medication or special diets then being followed by the particular patient, and other factors which those skilled in the art will recognize, with the appropriate dosage being at the discretion of the attending physician. Dosage regimes may be adjusted to provide the improved therapeutic response. An effective amount is also one in which any toxic or detrimental effects of the components are outweighed by the therapeutically beneficial effects.

The disclosed compounds may be prepared in the form of pharmaceutically acceptable salts. “Pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. These physiologically acceptable salts are prepared by methods known in the art, e.g., by dissolving the free amine bases with an excess of the acid in aqueous alcohol, or neutralizing a free carboxylic acid with an alkali metal base such as a hydroxide, or with an amine

The term “administering” means either directly administering a compound or composition as disclosed herein, or administering a prodrug, derivative or analog which will form an equivalent amount of the active compound or substance within the body.

The terms “subject,” “individual,” and “patient” are used interchangeably herein, and refers to a human or non-human animal to whom treatment, including prophylactic treatment, with the pharmaceutical composition according to disclosed subject matter, is provided. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys. In preferred embodiments, the subject is a human.

The term “antagonist” or “inhibitor” as used herein includes compounds that inhibit the expression or activity of a protein, polypeptide or enzyme and does not necessarily mean complete inhibition of expression and/or activity. Rather, the inhibition includes inhibition of the expression and/or activity of a protein, polypeptide or enzyme to an extent, and for a time, sufficient to produce the desired effect.

The term “peptidomimetic” as used herein includes compounds that are sterically similar to a reference compound and which mimic the activity of a peptide. Mimetics may include proteins or polypeptides. Peptidometics may also include non-peptides and/or may comprise amino acids linked by non-peptide bonds, e.g., without limitation, psi bonds. Mimetics may be conformationally constrained or cyclic.

Methods for treating intervertebral disc degeneration, promoting tissue regeneration in the intervertebral disc, promoting homeostasis of the intervertebral disc, preventing intervertebral disc degeneration and reducing inflammation of the intervertebral disc in a subject in need thereof, each method comprising administering a myeloid differentiation primary response (88) (MyD88) antagonist to the subject, are provided.

The myeloid differentiation primary response (88) (MyD88) antagonist can be administered to the subject by delivering the antagonist locally in the vicinity of the intervertebral disc. The myeloid differentiation primary response (88) (MyD88) antagonist can be coated onto or infused into an intervertebral device, biomaterial, or putty. These intervertebral devices, biomaterials, or putties can then be implanted, thus administering the myeloid differentiation primary response (88) (MyD88) antagonist. Representative devices can include bone plates, bone screws, pins and rods. These devices can be made from materials such as metals, plastics, ceramics, composites and silicone. Metals that can be used include stainless steel, cobalt-chromium alloys, titanium alloys, titanium, and tantalum. Plastics that can be used include polyethylene.

Inhibition of MyD88 signaling can be achieved by interfering with its TIR homodimerization domain using specific peptides that mimic the BB-loop. The consensus sequence for the BB loop is RDVLPGT (SEQ ID NO:22). Synthetic peptides comprising the sequence Ac-R-D-V-L-P-GT-NH₂ (SEQ ID NO: 23) are able to inhibit TIR domain-TIR domain interaction in MyD88. Epta peptides derived from the TIR domain of MyD88, for example, are effective in inhibiting homodimerization. (Loiarro et al., 2005).

In another embodiment, the myeloid differentiation primary response (88) (MyD88) antagonist can be delivered from an implantable device coated with a myeloid differentiation primary response (88) (MyD88) antagonist. The myeloid differentiation primary response (88) (MyD88) antagonist can be a nucleic acid, peptide, protein, antibody or antigen-binding fragment thereof In a preferred embodiment, the myeloid differentiation primary response (88) (MyD88) antagonist is a peptide. A preferred myeloid differentiation primary response (88) (MyD88) antagonist (e.g., a peptide) prevents homodimerization of MyD88. The peptide can comprise the amino acid sequence RDVLPGT (SEQ ID NO:22). In some embodiments, the peptide comprises the amino acid sequence DRQIKIWFQNRRMKWKKRDVLPGT (SEQ ID NO: 21). The underlined amino acid sequence is the MyD88 homodimerization sequence. The portion of the peptide that is not underlined is the protein transduction sequence, derived from the antennapedia protein, making the whole peptide permeable to the cell. In some embodiments, the peptide is MyD88i, a peptide having the amino acid sequence of SEQ ID NO: 21. In other embodiments, the peptide comprises the amino acid sequence RQIKIWFQNRRMKWKKRDVLPGTCVNS (SEQ ID NO: 24). The underlined amino acid sequence is the MyD88 homodimerization sequence. Without wishing to be bound by any mechanism of action, it is believed that this peptide functions by interfering with MyD88 homodimer formation.

In another embodiment, the myeloid differentiation primary response (88) (MyD88) antagonist is a peptidomimetic. In some embodiments, the peptidomimetic comprises (ST2825) described in WO 200606709. ST2825 was modeled on the structure of the consensus peptide in the BB-loop of the TIR of MyD88. (Loiarro et al., 2007). Without wishing to be bound by any mechanism of action, it is believed that this peptidomimetic functions by interfering with MyD88 homodimer formation.

In another embodiment, the myeloid differentiation primary response (88) (MyD88) antagonist is a small molecule mimetic. In some embodiments, the small molecule mimetic can be Compound 1 or a dimer of Compound 1 (EM-163). (Kissner et al., 2012). Without wishing to be bound by any mechanism of action, it is believed that these small molecules function by interfering with MyD88 homodimer formation.

Alternatively, the myeloid differentiation primary response (88) (MyD88) antagonist can be an RNAi agent. Representative RNAi agents include siRNA, shRNA, miRNA, dsRNA or ribozyme or variants thereof.

In other embodiments, a device, biomaterial or putty for implantation in or near the spine, that is coated or infused with a myeloid differentiation primary response gene (88) (MyD88) antagonist are provided. The device, biomaterial or putty can be for implantation in an intervertebral space.

Representative devices can include bone plates, bone screws, pins and rods. These devices can be made from materials such as metals, plastics, ceramics, composites and silicone. Metals that can be used include stainless steel, cobalt-chromium alloys, titanium alloys, titanium, and tantalum. Plastics that can be used include polyethylene.

In another embodiment, the myeloid differentiation primary response (88) (MyD88) antagonist to be coated on or infused in the device, biomaterial or putty can be a nucleic acid, peptide, protein, antibody or antigen-binding fragment thereof. In a preferred embodiment, the myeloid differentiation primary response (88) (MyD88) antagonist is a peptide. A preferred myeloid differentiation primary response (88) (MyD88) antagonist (e.g., a peptide) prevents homodimerization of MyD88. The peptide can comprise the amino acid sequence RDVLPGT (SEQ ID NO: 22). In some embodiments, the peptide comprises the amino acid sequence DRQIKIWFQNRRMKWKKRDVLPGT (SEQ ID NO: 21). In some embodiments, the peptide is MyD88i, a peptide having the amino acid sequence of SEQ ID NO: 21. Alternatively, the myeloid differentiation primary response (88) (MyD88) antagonist to be coated on or infused in the device, biomaterial or putty can be an RNAi agent. Representative RNAi agents include siRNA, shRNA, miRNA, dsRNA or ribozyme or variants thereof.

The major new findings from this study reveal the potent anti-inflammatory and anti-catabolic effects of MyD88 pathway inhibition on IVD homeostasis, suggesting a potential therapeutic benefit of inhibition of the MyD88 pathway in degenerative disc disease. In the disc, a MyD88 antagonist antagonizes LPS- and/or IL-1-mediated induction of cartilage-degrading enzymes, including MMP-1, MMP-13, ADAMTS-4, and ADAMTS-5, revealing a profound anti-catabolic role of such antagonists in NP cells from human as well as bovine tissues. Finally, a MyD88 antagonist counteracts IL-1-mediated suppression of PG deposition in mouse discs, suggesting that inhibition of MyD88 pathway-induced anti-catabolic effects in discs may be generalizable across different species both in vitro and ex vivo.

Inhibition of the MyD88 pathway also suppresses the catabolic factor-mediated upregulation of iNOS and TLR-2, elucidating anti-oxidative, anti-inflammatory, and possibly anti-pain effects in disc homeostasis. Interestingly, the in vitro cell culture studies using human astroglial cells demonstrate that inhibition of MyD88 by a MyD88 antagonist effectively attenuates both LPS- and IL-1-induced iNOS and inflammatory cytokines (e.g., TNFα, MCP-1) (data not shown) that may further activate juxtaposed glial cell population (e.g., microglial), potentially influencing nociceptive pathways after peripheral tissue damage (disc degeneration). The results consistently demonstrate a more potent capability of a MyD88 antagonist to antagonize the effects of LPS (p<0.01) compared to the powerful inflammatory cytokine IL-1 (p<0.05). The antagonistic potential of a MyD88 antagonist on LPS-mediated activity may have significant potential with profound beneficial effects in the human body. First, LPS is a major component of the cell membrane of gram-negative bacteria, serving as a crucial target for the development of new antimicrobial agents. LPS induces a variety of cellular responses in vivo involving septic shock, and humans are known to be much more sensitive to LPS than other animals (e.g., mice, rat, rabbit etc). Thus, the development of an anti-LPS medication (i.e., a MyD88 antagonist, such as MyD88i) may offer potential immunological benefits in fighting against unwanted endotoxemia. Second, as demonstrated in this study, inhibition of LPS-mediated effects via blockade of the MyD88 pathway may prevent or arrest joint degeneration in articular cartilage or discs. Upregulation of MMPs and ADAMTS after treatment with LPS or IL-1 in NP tissue as demonstrated herein reveals a striking ability of a MyD88 antagonist (e.g., MyD88i) to inhibit the LPS- and IL-1-mediated upregulation of matrix-degrading enzyme gene expression, protein expression, and activity levels in bovine and human discs, revealing similar anti-inflammatory effects as LfcinB in spine tissue.

In summary, it has been shown that MyD88 pathway-specific inhibition exerts potent anti-inflammatory and anti-catabolic effects in vitro and ex vivo via the inhibition of LPS- or IL-1-mediated activity in bovine and human disc cells. Together with the results from ex vivo organ culture studies, the data elucidate a possible role of MyD88 antagonists as described above, such as MyD88i, in tissue engineering to help relieve degenerative disc disease in the future.

In the following Examples, all parts and percentages are by weight, unless otherwise stated. It should be understood that these examples, while indicating preferred embodiments of the subject matter disclosed herein, are given by way of illustration only. From the above discussion and these examples, one skilled in the art can make various changes and modifications to adapt the disclosed subject matter to various usages and conditions.

### Examples

### Example 1

### Intervertebral Disc Cell Isolation and Culture

Human spine segments (T11/12 to S1) were received within 24 hours of death of donors from the Gift of Hope Organ and Tissue Donor Network (Elmhurst, Ill.), after obtaining consent from the families. Each of the lumbar segments underwent magnetic resonance imaging (MRI). Intact disc specimens (Thompsons grade 2 or 3) were removed and processed aseptically (Thompson et al., 1990; Kim et al., 2009). After dissection of the NP portion of discs either from human or bovine tails, NP cells were released by enzymatic digestion in Dulbecco's modified Eagle's medium [(DMEM)/Ham's F-12 (1:1)] culture medium with sequential treatments of 0.2% pronase and 0.025% collagenase P, as previously described (Kim et al.; Loeser et al., 2003). Alginate beads and monolayers were prepared for long-term (21 days) and short-term studies (1-2 days) as previously described by our group (Kim et al.; Im et al., 2003; Im et al., 2007b; Im et al., 2008; Li et al., 2008a).

For short-term monolayer cultures, isolated NP cells from either bovine tails or human spine discs were counted and plated onto 12-well plates at 8×10⁵ cells/cm² as previously described (Im et al., 2003; Im et al., 2007b). Cells were stimulated with either IL-1 (10 ng/mL, NCI) or LPS (10 μg/mL, Sigma-Aldrich, St. Louis, Mont.) in the presence or absence of MyD88 peptide inhibitor (MyD88i; IMGENEX, San Diego, Calif.) at the concentration of 100 μM, in 1 mL per well of serum-free medium for 24 hours at 37° C. under 5% CO₂. Supernatants were collected 24 hours after the initiation of each treatment and subjected to western blotting analyses for secreted matrix-degrading enzyme production. Cells were harvested and prepared for either cell lysates or total RNA extraction followed by real-time qPCR analyses, as previously described (Li et al., 2008b). 18s rRNA was used for normalization.

### Example 2

### Real-Time qPCR (Quantitative Polymerase Chain Reaction)

Total RNA was isolated from the cells using Trizol reagent (Invitrogen, Carlsbad, Calif.) following the instructions provided by the manufacturer. Reverse transcription was carried out with 1 μg total RNA using the ThermoScript™ RT-PCR system (Invitrogen, Carlsbad, Calif.) for first strand cDNA synthesis.

For real-time qPCR, cDNA was amplified using MyiQ Real-Time qPCR Detection System (Bio-Rad Hercules, Calif.). The synthesized cDNA was subjected to real-time qPCR utilizing the Bio-Rad iQTM SYBR Green supermix (Bio-Rad Hercules, Calif.). A threshold cycle (Ct value) was obtained from each amplification curve using iQ5 Optical System Software provided by the manufacturer (Bio-Rad Hercules, CA). Relative mRNA expression was determined using the ^{ΔΔ}C_{T} and calculated as follows: ^{ΔΔC}_{T}=^{ΔC}_{T} (gene of interest)−^{Δ}C_{T} (endogenous control). Gene quantification was conducted for entire, individual treatment groups and expressed in terms of fold change from untreated control levels. Expression of 18s rRNA was used as the internal control. The deviations in samples represent 3 different donors in 3 separate experiments. The primer sequences and their conditions for use are summarized in Table 1.

TABLE 1
shows the primer sequences used for qRT-PCR.

| | | NCBI |
|---|---|---|
| Gene | Primer sequences | accession No. |
| Bovine MMP-13 | SEQ ID NO: 1 Forward: 5′- | NM_174389.2 |
| | ACCCTTCCTTATCCCTTGATGCCA-3′ | |
| | SEQ ID NO: 2 Reverse: 5′- | |
| | AAACAGCTCTGCTTCAACCTGCTG-3′ | |
| Bovine ADAMTS-4 | SEQ ID NO: 3 Forward: 5′- | NM_181667.1 |
| | AGTTCGACAAGTGCATGGTGTGTG-3′ | |
| | SEQ ID NO: 4 Reverse: 5′- | |
| | ATGGTGACCACGTTGTTGTATCCGT-3′ | |
| Bovine ADAMTS-5 | SEQ ID NO: 5 Forward: 5′- | NM_001166515.1 |
| | TCACCAGCATTGACGCATCCAAAC-3′ | |
| | SEQ ID NO: 6 Reverse: 5′- | |
| | TTCGTGGTAGGTCCAGCAAACAGT-3′ | |
| Bovine TLR-2 | SEQ ID NO: 7 Forward: 5′- | AF368419 |
| | TAAGGATGCCTGGCCCTTCCTTCAAA-3′ | |
| | SEQ ID NO: 8 Reverse: 5′- | |
| | GGCCACTGACAAGTTTCAGGCATT-3′ | |
| Human MMP-13 | SEQ ID NO: 9 Forward: 5′- | NM_002427.2 |
| | ACCCTGGAGCACTCATGTTTCCTA-3′ | |
| | SEQ ID NO: 10 Reverse: 5′- | |
| | TGGCATCAAGGGATAAGGAAGGGT-3′ | |
| Human MMP-1 | SEQ ID NO: 11 Forward: 5′- | NM_002421.2 |
| | CATCCTGTTGTTGCTGTGGCTGAT-3′ | |
| | SEQ ID NO: 12 Reverse: 5′- | |
| | GCGTGGATGCCGCATATGAAGTTA-3′ | |
| Human ADAMTS-4 | SEQ ID NO: 13 Forward: 5′- | NM_005099.4 |
| | AGTTTGAATGGGCCTTTGAGCACC-3′ | |
| | SEQ ID NO: 14 Reverse: 5′- | |
| | TAGTCCTTGCCAGGGAAAGTCACA-3′ | |
| Human iNOS | SEQ ID NO: 15 Forward: 5′- | NM_000625.4 |
| | 5′-ATCACACGCCCACAGAGATCCA-3′ | |
| | SEQ ID NO: 16 Reverse: 5′- | |
| | GCTTCAGGCTGTTGAGCCATGT-3′ | |
| Bovine 18s rRNA | SEQ ID NO: 17 Forward: 5′- | NR_003286 |
| | GTAACCCGTTGAACCCCATT-3′ | |
| | SEQ ID NO: 18 Reverse: 5′- | |
| | CCATCCAATCGGTAGTAGCG-3′ | |
| Human 18s rRNA | SEQ ID NO: 19 Forward: 5′- | NR_003286 |
| | ATCCATTGGAGGGCAAGTCTGGT-3′ | |
| | SEQ ID NO: 20 Reverse: 5′- | |
| | GCTCCCAAGATCCAACTACGAGCTTT-3′ | |

### Example 3

### Immunoblotting

Cell and tissue lysates were prepared using modified RIPA buffer, as previously described (Li et al., 2008a; Li et al., 2008c). The total protein concentrations of cell lysates were determined by a bicinchoninic acid protein assay (Pierce, Rockford, Ill., USA). An equal amount of protein was resolved by 10% SDS-polyacrylamide gels (SDS-PAGE) and transferred to a nitrocellulose membrane for immunoblot analyses, as described previously (Im et al., 2007b). MMP-13 and MMP-1 antibodies were provided by courtesy of Dr. Gillian Murphy (Cambridge University, UK) Immunoreactivity was visualized using the ECL system (Amersham Biosciences, Piscataway, N.J., USA) and the Signal Visual Enhancer system (Pierce), which magnifies the signal.

### Example 4

### Ex vivo Analysis Using an Intradiscal Injection Organ Culture Model

Lumbar spine segments from deceased mice were dissected under sterile conditions within 12 hrs after sacrifice at the Rush University animal facility for ex vivo organ culture, as previously described (Chiba et al., 1998). The requirement for the pre-treatment of MyD88 has been previously described (Ahmad et al., 2007). Therefore, intradiscal pre-injection of MyD88i (150 μ per disc) en bloc was performed using a 30-gauge needle (30 G, 1.50 μL volume). Injected discs were then separated and incubated in DMEM/Ham's F-12 medium supplemented with 1% mini-ITS. After 24 hrs, the MyD88i pre-injected discs were challenged with either IL-1 (100 ng/mL) or LPS (10 μg/mL) and further incubated for 6 days. Harvested discs were fixed in 4% paraformaldehyde and then decalcified in EDTA, which was changed every 5 days. The decalcified discs were paraffin embedded. Serial disc sections of exactly 5-μm thickness were obtained to prepare slides. Safranin O-fast green staining was performed to assess general morphology and the loss of PG in disc ground substance.

On the last day of organ culture, the harvested mouse lumbar disc cells were assessed to evaluate cell viability with fluorescent microscopy using the LIVE/DEAD™ Viability/Cytotoxicity Kit (Molecular Probes, Eugene, Oreg.) by modifying previously described methods (Del Carlo and Loeser, 2002; Junger et al., 2009). Briefly, sample disc tissues were dissected out and cells were isolated via enzymatic digestion (sequential treatments with pronase and collagenase). The cells were then incubated in serum free medium supplemented with 10 μM calcein AM green and 1 μM ethidium homodimer-1 for 30 min. Membrane-permeable calcein AM is cleaved by esterases in live cells to yield cytoplasmic green fluorescence, and membrane-impermeable ethidium homodimer-1 labels nucleic acids of membrane-compromised cells with red fluorescence. At least 100 cells were counted in triplicate for each data point.

### Example 5

### Histologic Analysis of Injected Discs

Harvested discs were fixed in 4% paraformaldehyde and decalcified in EDTA, which was changed every 5 days. The decalcified discs were paraffin embedded. Serial disc sections of exactly 5-μm thickness were obtained to prepare slides. Safranin O-fast green staining was performed to assess general morphology and the loss of PG in disc ground substance, as previously described (Muddasani et al., 2007). All samples from discs that were stained were examined independently by two blinded observers.

### Example 6

### Gelatin Zymography

Gelatin zymography was then performed as previously described (Gupta et al., 2007). Briefly, an equal volume of cell culture supernatant was mixed with non-reducing sample buffer [4% SDS, 0.15 M Tris (pH 6.8), and 20% (volume/volume) glycerol containing 0.05% (weight/volume) bromophenol blue] and resolved on a 10% polyacrylamide gel containing copolymerized 0.2% (1 mg/mL) swine skin gelatin (Sigma). After electrophoresis of the conditioned medium supernatant samples, gels were washed twice, for 15 minutes each time, with 2.5% Triton X-100. Digestion was carried out by incubating the gel in the gelatinase buffer (50 mM Tris-HCl (pH 7.6), 10 mM CaCl₂, 50 mM NaCl, and 0.05% Brij-35) at 37° C. for 24 hours. The gel was stained with 0.1% Coomassie brilliant blue R350 (GE Healthcare, Piscataway, N.J., USA), and the locations of gelatinolytic activity were revealed as clear bands on a background of uniform light blue staining. After development, gel images were captured and the clear bands were analyzed using “ImageJ” image analysis software (www.imagej.nih.gov), and were expressed in arbitrary optical density units. Data shown are cumulative of two experiments. P-values presented as mean ±standard deviation; data without a common letter differ, P<0.05.

### Example 7

### Statistical Analysis

Analysis of variance was performed using StatView 5.0 software (SAS Institute, Cary, N.C.). P-values lower than 0.05 were considered significant.

### Example 8

### Inhibition of The MyD88 Pathway Suppresses LPS- and IL-1-induced Expression of Matrix-Degrading Enzymes and TLR-2 in Both Bovine and Human NP cells

LPS and the inflammatory cytokine IL-1 both induce catabolic effects in cartilage via an upregulation of matrix-degrading enzymes such as MMP-1 and MMP-13, which are key matrix-degrading enzymes in articular cartilage as well as in the IVD (Martel-Pelletier et al., 2001; Le Maitre et al., 2004; Le Maitre et al., 2007). Similar to collagenases, members of the ADAMTS family (i.e., aggrecanases) induce cartilage degradation as well. Specifically, upregulation of ADAMTS-4 and -5 has been correlated with degradation of aggrecan (major component of PGs) in the IVD, ultimately resulting in disc degeneration (Martel-Pelletier et al., 2001; Le Maitre et al., 2004; Le Maitre et al., 2007). Therefore, the capacity for MyD88i to inhibit catabolic factor-mediated upregulation of aggrecanase expression in NP cells was assessed.

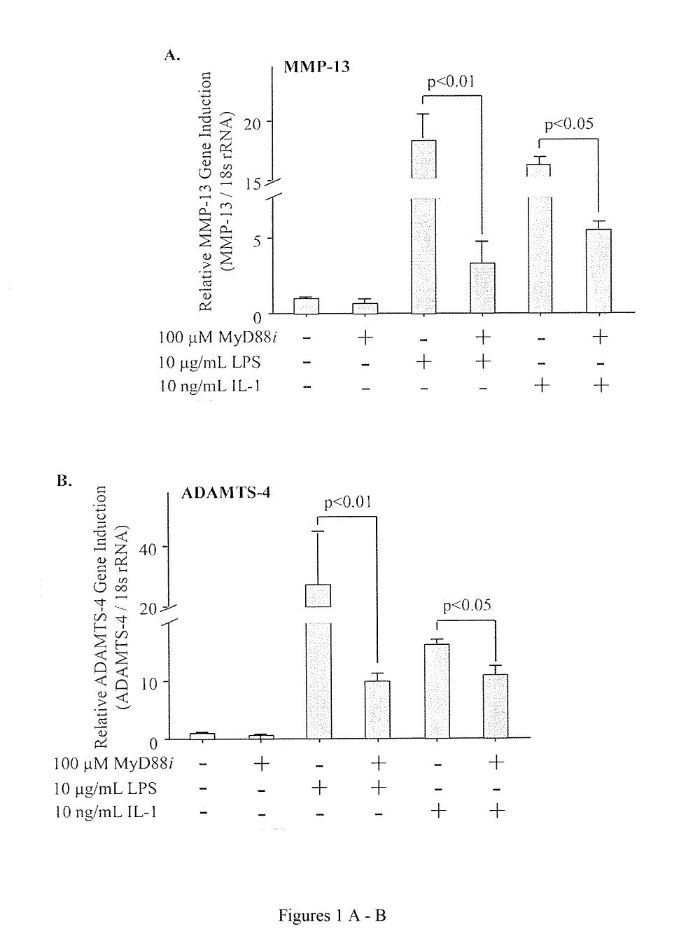

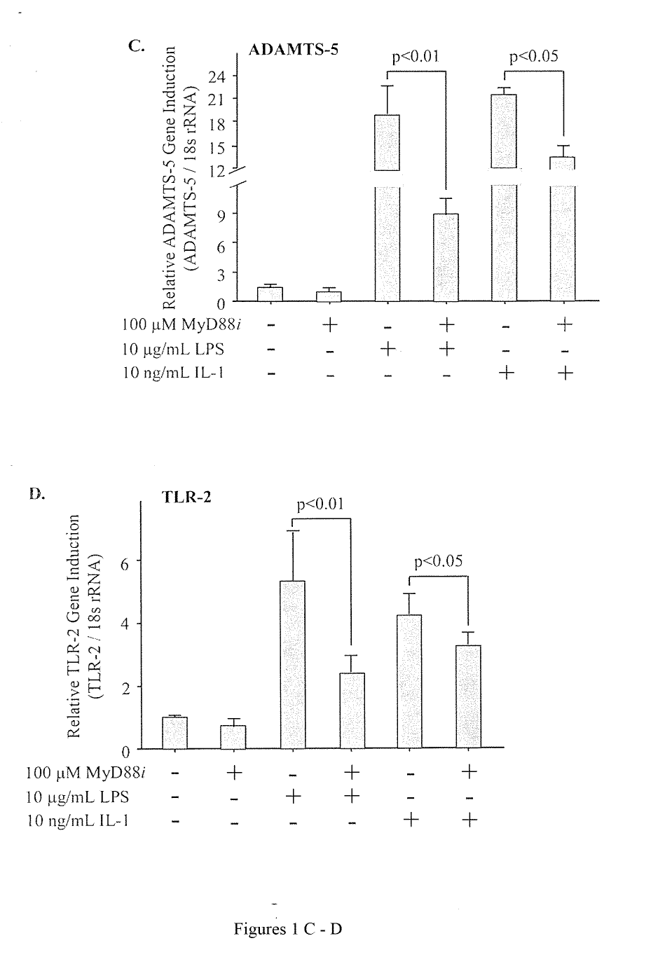

Given that MyD88 is the key regulatory cofactor that transduces TLR signals to induce matrix-degrading enzyme expression and ECM degradation in articular chondrocytes (Liu-Bryan and Terkeltaub, 2010; Abdollahi-Roodsaz et al., 2011), the potential for MyD88i to antagonize catabolic effects induced by LPS and IL-1 in bovine IVD cells was examined In initial experiments using monolayer cultures with different concentrations of peptide inhibitor of MyD88 (MyD88i), it was found that that 100 μM of MyD88i is a minimal dose demonstrating maximal effects. Therefore, NP cells were treated with MyD88i (100 μM), LPS or IL-1, individually, or combinations of LPS and MyD88i or IL-1 and MyD88i for 24 hours. Resulting cellular responses were then analyzed for alterations in gene expression and/or secreted protein levels. Our data reveal the significant upregulation of collagenase (MMP-13) and aggrecanases (ADAMTS-4, ADAMTS-5) after stimulation with either LPS or IL-1 compared to control (untreated) in bovine NP cells (FIGS. 1A˜C). Treatment of cells with MyD88i significantly suppresses either LPS- or IL-1-induced upregulation of catabolic gene expression (p<0.01 and p<0.05, respectively).

It is believed that TLR-2 is an essential component to the development of arthritis in peripheral joints, and is associated with upregulation of pro-inflammatory cytokines and destructive tissue enzymes in articular cartilage (Joosten et al., 2003; Kim et al., 2006). Treatment of NP cells with LPS or IL-1 leads to an upregulation of TLR-2 suggesting an auto- and paracrine-regulatory mechanism of TLR-2 pathway by inflammatory ligands (FIG. 1D). Pretreatment of these cells with MyD88i, however, appears to attenuate this auto- and paracrine-loop via a significant decrease in LPS- or IL-1-stimulated TLR-2 levels (p<0.01 and p<0.05, respectively). These data suggest MyD88i may antagonize the inflammatory response via downregulation of TLR-2.

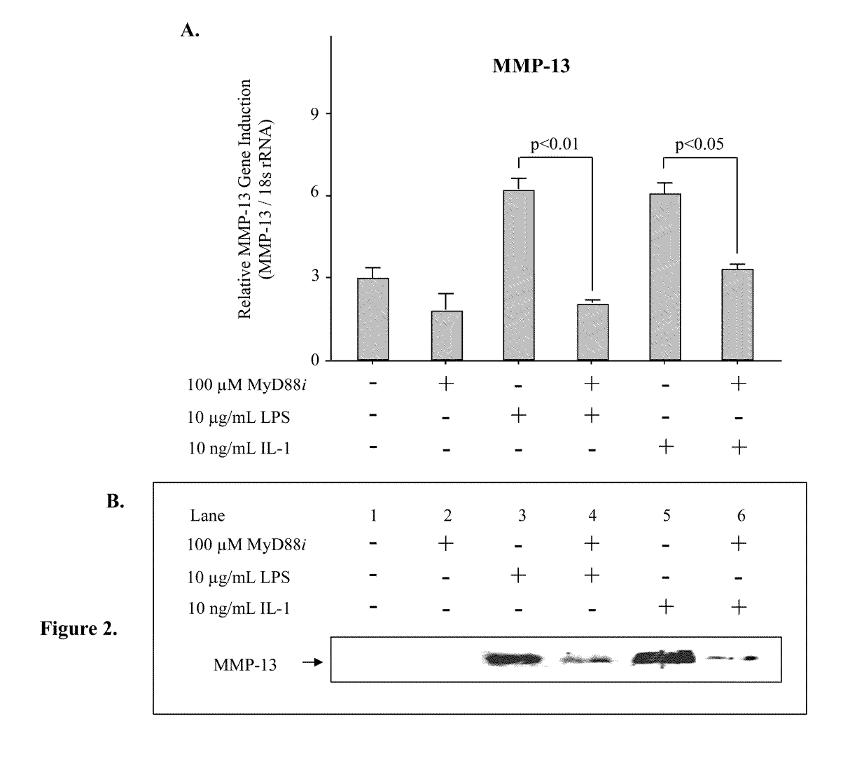

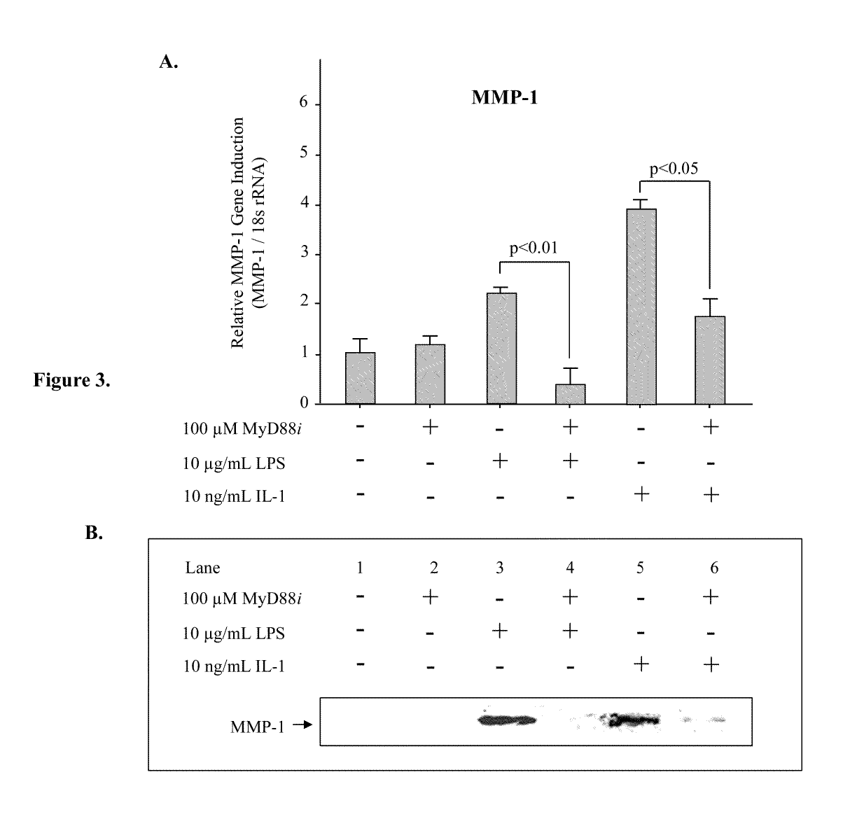

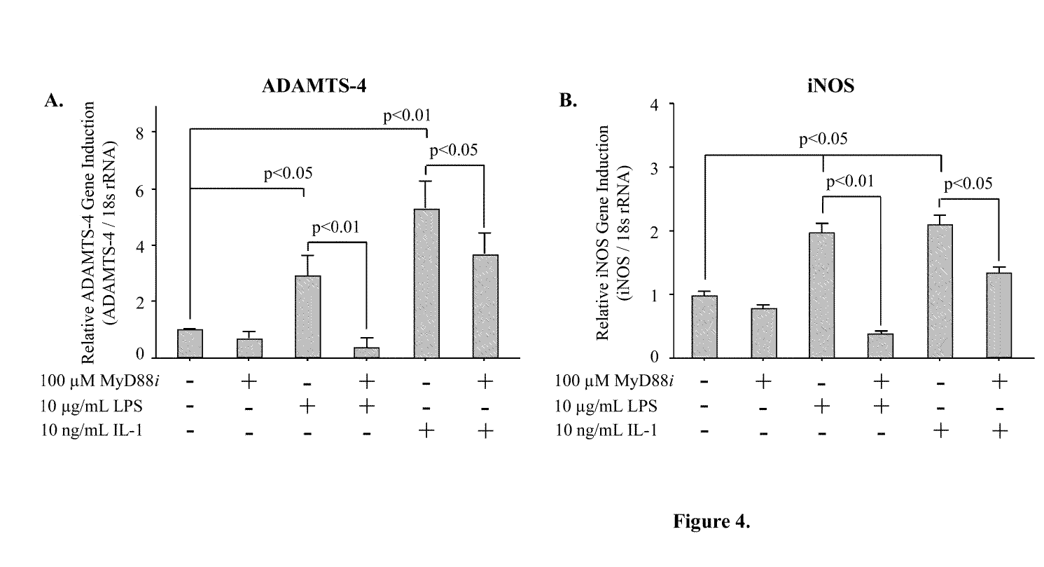

Our data generated using bovine tail disc NP cells were further verified using human lumbar disc NP cells (FIGS. 2˜4). Similar to the cellular response in bovine NP cells, human NP cells reveal an increase in collagenases such as MMP-13 (FIGS. 2A & 2B) and MMP-1 (FIGS. 3A & 3B) at both mRNA and protein levels after stimulation with LPS or IL-1, with significant decreases in these catabolic gene expressions by co-treatment with MyD88i (p<0.01 for LPS plus MyD88i and p<0.05 for IL-1 plus MyD88i, respectively). Similarly, induction of ADAMTS-4 expression by LPS (p<0.05) or IL-1 (p<0.01) is significantly reduced by co-treatment with MyD88i in human NP cells (FIG. 4A; p<0.01, p<0.05, respectively). These data demonstrate potent anti-catabolic activity of MyD88i in both bovine and human NP cells in vitro, suggesting an important role of inhibition of MyD88 to limit ECM degradation in discs.

Nitric oxide (NO) is known to play an important role in cartilage metabolism and has been linked to inflammatory pathways and pain signaling (Kohyama et al., 2000; Del Carlo and Loeser, 2002; Castro et al., 2006). Increased expression of nitric oxide synthase (iNOS), a gene responsible for the production of NO, has been associated with cartilage destruction in knee joints (Pelletier et al., 1999) and IVDs (Kohyama et al., 2000), and a reduction of iNOS is closely linked to a reduction in tissue levels of multiple MMPs (Pelletier et al., 1999). We therefore examined the role of MyD88 inhibition in LPS- or IL-1-regulation of iNOS gene in human NP cells (FIG. 4B). After stimulation with LPS or IL-1, iNOS mRNA is significantly increased (p<0.05; 2-fold) compared to control (untreated). Co-treatment with MyD88i, however, results in significant suppression of iNOS gene levels (p<0.01 for LPS; p<0.05 for IL-1, respectively) compared to treatment with LPS or IL-1 alone, suggesting the role of MyD88 inhibition in an oxidative stress-related cellular response in disc cells.

### Example 9

### MyD88 Peptide Inhibitor Antagonizes LPS- or IL-1-induced Catabolic Protease Activity in the IVD

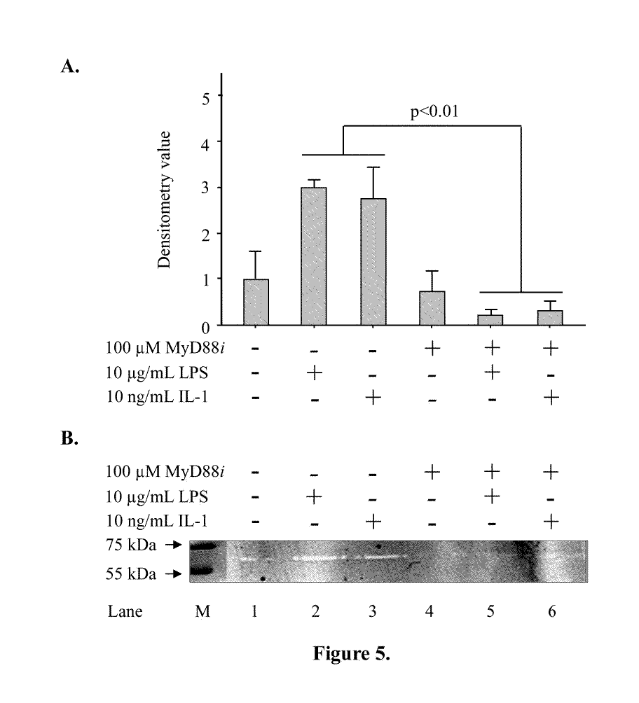

Because MyD88i effectively suppresses expression and/or production of multiple cartilage degrading enzymes, TLR-2 and stress-associated genes after stimulation with either LPS or IL-1 (FIGS. 1˜4), the role of MyD88i in antagonizing catalytic enzyme activity was further analyzed by performing reverse zymography (FIG. 5). Human NP cells cultured in monolayer were stimulated with either LPS or IL-1 in the presence or absence of MyD88i, followed by zymographic analyses. Our results reveal that stimulation of NP cells with either LPS or IL-1 enhances catalytic activity up to 2-fold compared to control level (untreated) assessed by densitometry value (FIG. 5A; lanes 2 & 3), as represented by hydrolyzed clear bands of gelatin gel (FIG. 5B; lanes 2 & 3). Importantly, addition of MyD88i inhibits the catabolic effects mediated by either LPS or IL-1, as catalytic enzyme activity is markedly suppressed by the presence of MyD88i (p<0.01; lanes 5 & 6). Taken together, these findings demonstrate the potent capacity of MyD88i to antagonize catabolic enzyme activity, in addition to its suppressive effects on mRNA and protein expression.

### Example 10

### MyD88 Inhibition Antagonizes Inflammatory Cytokine-Mediated Proteoglycan Depletion in Mouse Disc Organ Culture Model

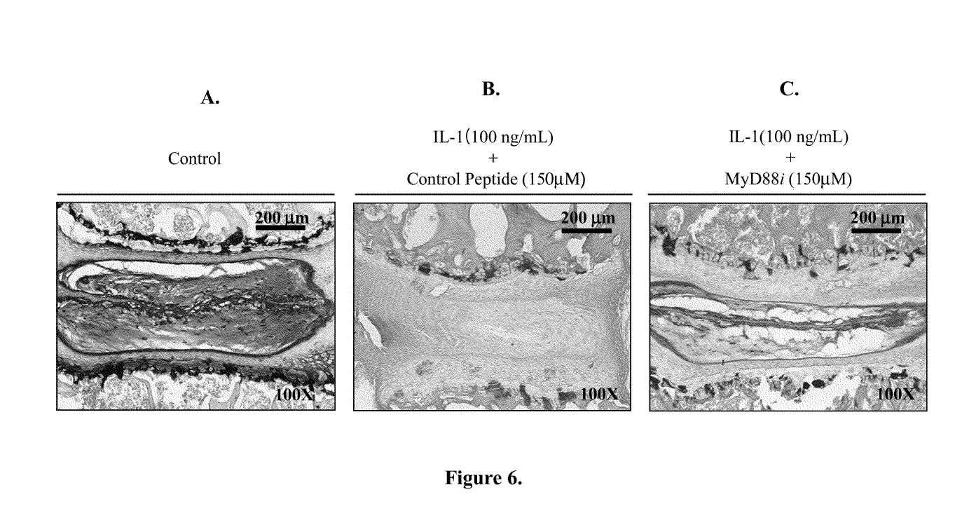

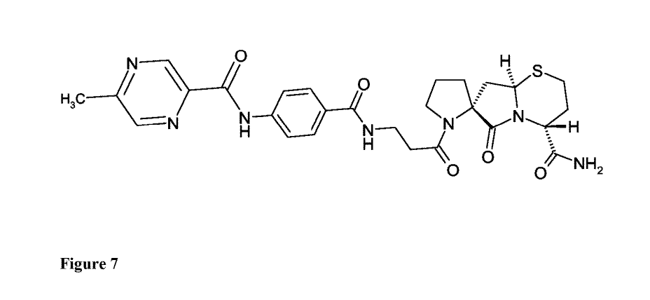

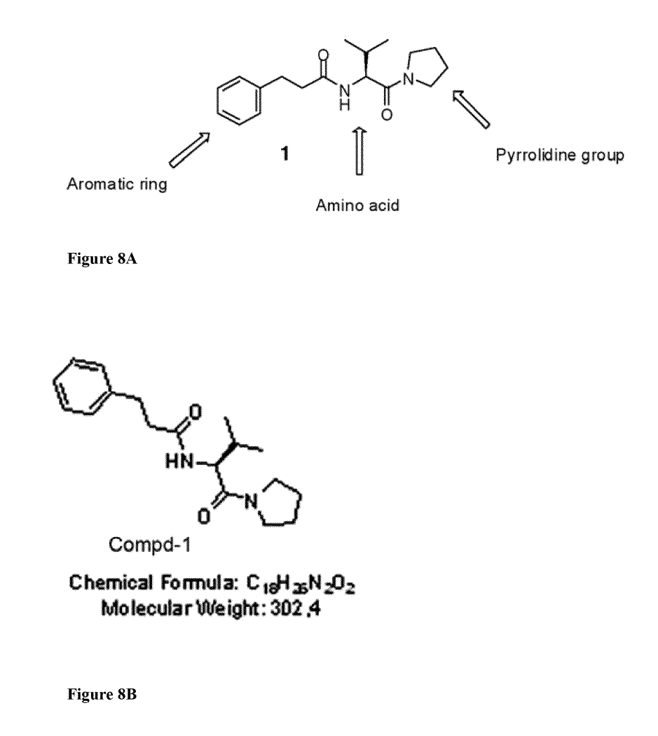

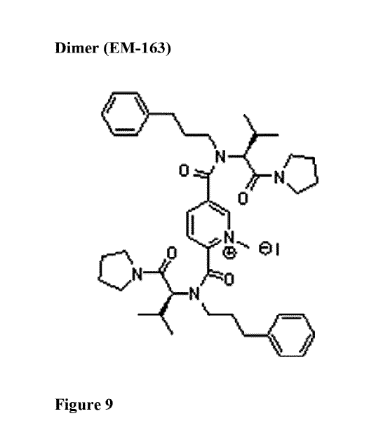

Given the in vitro results obtained above, the potential physiologic effects of MyD88i were tested in an organ culture model. Mouse discs were intradiscally injected en bloc with either MyD88i (150 μM per disc) or control peptide (no cell permeability sequence, 150 μM per disc) followed by subsequent stimulation with IL-1 (100 ng/mL) in culture media. After 6 days of lumbar disc organ culture, the presence of IL-1 with control peptide reveals severe PG depletion (FIG. 6B) compared to co-incubation with MyD88i (FIG. 6C) or control (FIG. 6A). The ex vivo experiment results using a disc organ culture system suggest that injection of MyD88i significantly attenuates the PG loss that was induced by a potent inflammatory cytokine IL-1 and maintains the integrity of disc matrix homeostasis.

### References

Abdollahi-Roodsaz, S., van de Loo, F. A., Koenders, M. I., Helsen, M. M., Walgreen, B., van den Bersselaar, L. A., Arntz, O. J., Takahashi, N., Joosten, L. A. and van den Berg, W. B. Destructive role of myeloid differentiation factor 88 and protective role of TIR-containing adaptor inducing interferon beta in IL-17-dependent arthritis. Arthritis Rheum (2011).
Ahmad, R., Sylvester, J. and Zafarullah, M. MyD88, IRAK1 and TRAF6 knockdown in human chondrocytes inhibits interleukin-1-induced matrix metalloproteinase-13 gene expression and promoter activity by impairing MAP kinase activation. Cell Signal 19 (2007), pp. 2549-57.
Andersson, G. B. Epidemiological features of chronic low-back pain. Lancet 354 (1999), pp. 581-5.
Aota, Y., An, H. S., Imai, Y., Thonar, E. J., Muehleman, C. and Masuda, K. Comparison of cellular response in bovine intervertebral disc cells and articular chondrocytes: effects .of lipopolysaccharide on proteoglycan metabolism. Cell Tissue Res 326 (2006), pp. 787-93.
Bobacz, K., Sunk, I. G., Hofstaetter, J. G., Amoyo, L., Toma, C. D., Akira, S., Weichhart, T., Saemann, M. and Smolen, J. S. Toll-like receptors and chondrocytes: the lipopolysaccharide-induced decrease in cartilage matrix synthesis is dependent on the presence of toll-like receptor 4 and antagonized by bone morphogenetic protein 7. Arthritis Rheum 56 (2007), pp. 1880-93.
Carminati, P., Gallo, G., Ruggiero, V., Sassano, M., Mastroianni, D. MyD88 homodimerization inhibitors, Pub. No. WO20006067091.
Castro, R. R., Cunha, F. Q., Silva, F. S., Jr. and Rocha, F. A. A quantitative approach to measure joint pain in experimental osteoarthritis--evidence of a role for nitric oxide. Osteoarthritis Cartilage 14 (2006), pp. 769-76.
Chiba, K., Andersson, G. B., Masuda, K., Momohara, S., Williams, J. M. and Thonar, E. J. A new culture system to study the metabolism of the intervertebral disc in vitro. Spine (Phila Pa. 1976) 23 (1998), pp. 1821-7; discussion 1828.
Crean, J. K., Roberts, S., Jaffray, D. C., Eisenstein, S. M. and Duance, V. C. Matrix metalloproteinases in the human intervertebral disc: role in disc degeneration and scoliosis. Spine (Phila Pa 1976) 22 (1997), pp. 2877-84.
Del Carlo, M., Jr. and Loeser, R. F. Nitric oxide-mediated chondrocyte cell death requires the generation of additional reactive oxygen species. Arthritis Rheum 46 (2002), pp. 394-403.

Freemont, T. J., LeMaitre, C., Watkins, A. and Hoyland, J. A. Degeneration of intervertebral discs: current understanding of cellular and molecular events, and implications for novel therapies. Expert Rev Mol Med 2001 (2001), pp. 1-10.
Goldring, M. B. The role of the chondrocyte in osteoarthritis. Arthritis Rheum 43 (2000), pp. 1916-26.
Gupta, K., Shukla, M., Cowland, J. B., Malemud, C. J. and Haqqi, T. M. Neutrophil gelatinase-associated lipocalin is expressed in osteoarthritis and forms a complex with matrix metalloproteinase 9. Arthritis Rheum 56 (2007), pp. 3326-35.
Iatridis, J. C., Setton, L. A., Weidenbaum, M. and Mow, V. C. Alterations in the mechanical behavior of the human lumbar nucleus pulposus with degeneration and aging. J Orthop Res 15 (1997), pp. 318-22.
Im, H. J., Li, X., Muddasani, P., Kim, G. H., Davis, F., Rangan, J., Forsyth, C. B., Ellman, M. and Thonar, E. J. Basic fibroblast growth factor accelerates matrix degradation via a neuro-endocrine pathway in human adult articular chondrocytes. J Cell Physiol (2007a).
Im, H. J., Li, X., Muddasani, P., Kim, G. H., Davis, F., Rangan, J., Forsyth, C. B., Ellman, M. and Thonar, E. J. Basic fibroblast growth factor accelerates matrix degradation via a neuro-endocrine pathway in human adult articular chondrocytes. J Cell Physiol 215 (2008), pp. 452-63.
Im, H. J., Muddasani, P., Natarajan, V., Schmid, T. M., Block, J. A., Davis, F., van Wijnen, A. J. and Loeser, R. F. Basic fibroblast growth factor stimulates matrix metalloproteinase-13 via the molecular cross-talk between the mitogen-activated protein kinases and protein kinase Cdelta pathways in human adult articular chondrocytes. J Biol Chem 282 (2007b), pp. 11110-21.
Im, H. J., Pacione, C., Chubinskaya, S., Van Wijnen, A. J., Sun, Y. and Loeser, R F Inhibitory effects of insulin-like growth factor-1 and osteogenic protein-1 on fibronectin fragment- and interleukin-1beta-stimulated matrix metalloproteinase-13 expression in human chondrocytes. J Biol Chem 278 (2003), pp. 25386-94.
Jasin, H. E. Bacterial lipopolysaccharides induce in vitro degradation of cartilage matrix through chondrocyte activation. J. Clin Invest 72 (1983), pp. 2014-9.
Joosten, L. A., Koenders, M. I., Smeets, R. L., Heuvelmans-Jacobs, M., Helsen, M. M., Takeda, K., Akira, S., Lubberts, E., van de Loo, F. A. and van den Berg, W. B. Toll-like receptor 2 pathway drives streptococcal cell wall-induced joint inflammation: critical role of myeloid differentiation factor 88. J Immunol 171 (2003), pp. 6145-53.
Junger, S., Gantenbein-Ritter, B., Lezuo, P., Alini, M., Ferguson, S. J. and Ito, K. Effect of limited nutrition on in situ intervertebral disc cells under simulated-physiological loading. Spine (Phila Pa. 1976) 34 (2009), pp. 1264-71.
Kim, H. A., Cho, M. L., Choi, H. Y., Yoon, C. S., Shun, J. Y., Oh, H. J. and Kim, H. Y. The catabolic pathway mediated by Toll-like receptors in human osteoarthritic chondrocytes. Arthritis Rheum 54 (2006), pp. 2152-63.
Kim, J. S., Ellman, M. B., An, H. S., van Wijnen, A. J., Borgia, J. A. and Im, H. J. Insulin-like growth factor 1 synergizes with bone morphogenetic protein 7-mediated anabolism in bovine intervertebral disc cells. Arthritis Rheum 62, pp. 3706-15.
Kim, K. W., Chung, H. N., Ha, K. Y., Lee, J. S. and Kim, Y. Y. Senescence mechanisms of nucleus pulposus chondrocytes in human intervertebral discs. Spine J9 (2009), pp. 658-66.
Kissner, T., Ruthel, G., Alam, S., Mann, Enrique, Ajami, D., Rebek, M., Larkin, E., Fernandez, S., Ulrich, R., Ping, S., Waugh, D., Rebek, Jr., J., and Saikh, K. Therapeutic Inhibition of Pro-Inflammatory Signalling and Toxicity to Staphylococcal Enterotoxin B by a Synthetic Dimeric BB-Loop Mimetic of MyD88. PLoS ONE, 7 (2012), e40773, pp. 1-12.
Kohyama, K., Saura, R., Doita, M. and Mizuno, K. Intervertebral disc cell apoptosis by nitric oxide: biological understanding of intervertebral disc degeneration. Kobe J Med Sci 46 (2000), pp. 283-95.
Le Maitre, C. L., Freemont, A. J. and Hoyland, J. A. Localization of degradative enzymes and their inhibitors in the degenerate human intervertebral disc. J Pathol 204 (2004), pp. 47-54.
Le Maitre, C. L., Pockert, A., Buttle, D. J., Freemont, A. J. and Hoyland, J. A. Matrix synthesis and degradation in human intervertebral disc degeneration. Biochem Soc Trans 35 (2007), pp. 652-5.
Lee, S., Moon, C. S., Sul, D., Lee, J., Bae, M., Hong, Y., Lee, M., Choi, S., Derby, R., Kim, B. J., Kim, J., Yoon, J. S., Wolfer, L., Wang, J., Hwang, S. W. and Lee, S. H. Comparison of growth factor and cytokine expression in patients with degenerated disc disease and herniated nucleus pulposus. Clin Biochem 42 (2009), pp. 1504-11.
Li, X., An, H. S., Ellman, M., Phillips, F., Thonar, E. J., Park, D. K., Udayakumar, R. K. and Im, H.J. The action of fibroblast growth factor-2 on the intervertebral disc. Arthritis Res Ther 10 (2008a), p. R48.
Li, X., An, H. S., Ellman, M., Phillips, F., Thonar, E. J., Park, D. K., Udayakumar, R. K. and Im, H. J. Action of fibroblast growth factor-2 on the intervertebral disc. Arthritis Res Ther 10 (2008b), p. R48.
Li, X., Phillips, F. M., An, H. S., Ellman, M., Thonar, E. J., Wu, W., Park, D. and Im, H. J. The action of resveratrol, a phytoestrogen found in grapes, on the intervertebral disc. Spine (Phila Pa. 1976) 33 (2008c), pp. 2586-95.
Liu-Bryan, R. and Terkeltaub, R. Chondrocyte innate immune myeloid differentiation factor 88-dependent signaling drives procatabolic effects of the endogenous Toll-like receptor 2/Toll-like receptor 4 ligands low molecular weight hyaluronan and high mobility group box chromosomal protein 1 in mice. Arthritis Rheum 62 (2010), pp. 2004-12.
Loeser, R. F., Todd, M. D. and Seely, B. L. Prolonged treatment of human osteoarthritic chondrocytes with insulin-like growth factor-I stimulates proteoglycan synthesis but not proteoglycan matrix accumulation in alginate cultures. J Rheumatol 30 (2003), pp. 1565-70.
Loiarro, M., Sette, C., Gallo, G., Ciacci, A., Fanto, N., Mastroianni, D., Carminati, P., and Ruggiero, V. Peptide-mediated Interference of TIR Domain Dimerization in MyD88 Inhibits Interleukin-1-dependent Activation of NF-κB. J Biol Chem 280 (2005), pp.15809-814.
Loiarro, M., Capolunghi, F., Fanto, N., Gallo, G., Campo, S., Arseni, B., Carsetti, R., Carminati, P., De Santis, R., Ruggiero, V., and Sette, C. Pivotal Advance: Inhibition of MyD88 dimerization and recruitment of IRAK1 and IRAk4 by a novel peptidomimetic compound. J. of Leukocyte Biol 82 (2007), pp. 801-810.
Martel-Pelletier, J., Welsch, D. J. and Pelletier, J. P. Metalloproteases and inhibitors in arthritic diseases. Best Pract Res Clin Rheumatol 15 (2001), pp. 805-29.
Masuda, K., Imai Y., Okuma M., Muehleman C., Nakagawa K., Akeda K., Thonar E., Andersson G., An, HS. Osteogenic Protein-1 Injection Into a Degenerated Disc Induces the Restoration of Disc Height and Structural Changes in the Rabbit Anular Puncture Model. Spine 31 (2006), pp. 742-754.
Mooney, V. The classification of low back pain. Ann Med 21 (1989), pp. 321-5.
Muddasani, P., Norman, J. C., Ellman, M., van Wijnen, A. J. and Im, H. J. Basic fibroblast growth factor activates the MAPK and NFkappaB pathways that converge on Elk-1 to control production of matrix metalloproteinase-13 by human adult articular chondrocytes. J Biol Chem 282 (2007), pp. 31409-21.
Nomura, T., Mochida, J., Okuma, M., Nishimura, K. and Sakabe, K. Nucleus pulposus allograft retards intervertebral disc degeneration. Clin Orthop Relat Res (2001), pp. 94-101.
Pelletier, J. P., Lascau-Coman, V., Jovanovic, D., Fernandes, J. C., Manning, P., Connor, J. R., Currie, M. G. and Martel-Pelletier, J. Selective inhibition of inducible nitric oxide synthase in experimental osteoarthritis is associated with reduction in tissue levels of catabolic factors. J Rheumatol 26 (1999), pp. 2002-14.
Takeda, K. and Akira, S. Toll-like receptors in innate immunity. Int Immunol 17 (2005), pp. 1-14.
Takeda, K., Kaisho, T. and Akira, S. Toll-like receptors. Annu Rev Immunol 21 (2003), pp. 335-76.
Thompson, J. P., Pearce, R. H., Schechter, M. T., Adams, M. E., Tsang, I. K. and Bishop, P. B. Preliminary evaluation of a scheme for grading the gross morphology of the human intervertebral disc. Spine (Phila Pa. 1976) 15 (1990), pp. 411-5.

What is claimed:

1. A method for treating or preventing intervertebral disc degeneration in a subject in need thereof comprising administering a myeloid differentiation primary response (88) (MyD88) antagonist to the subject.

2. The method of claim 1 wherein said myeloid differentiation primary response (88) (MyD88) antagonist is delivered locally in the vicinity of the intervertebral disc.

3. The method of claim 2 wherein said administering comprises implanting an intervertebral device, biomaterial, or putty.

4. The method of claim 3 wherein the myeloid differentiation primary response (88) (MyD88) antagonist is delivered from an implantable device coated with a myeloid differentiation primary response (88) (MyD88) antagonist.

5. The method of claim 4 wherein said myeloid differentiation primary response (88) (MyD88) antagonist is a nucleic acid, peptide, protein, antibody or antigen-binding fragment thereof.

6. The method of claim 5 wherein the myeloid differentiation primary response (88) (MyD88) antagonist is a peptide.

7. The method of claim 6 wherein the peptide comprises the amino acid sequence of SEQ ID NO: 22.

8. The method of claim 7 wherein said myeloid differentiation primary response (88) (MyD88) antagonist prevents homodimerization of MyD88.

9. The method of claim 5 wherein the nucleic acid is an RNAi agent.

10. The method of claim 9 wherein the RNAi agent is siRNA, shRNA, miRNA, dsRNA or ribozyme or variants thereof

11. The method of claim 8 wherein the subject is a mammal

12. The method of claim 11 wherein the mammal is a human.

13. A device for implantation in or near the spine, wherein said device is coated or infused with a myeloid differentiation primary response gene (88) (MyD88) antagonist.

14. The device of claim 13 wherein said device is for implantation in an intervertebral space.

15. The device of claim 14 wherein said myeloid differentiation primary response (88) (MyD88) antagonist is a nucleic acid, peptide, protein, antibody or antigen-binding fragment thereof.

16. The device of claim 15 wherein the myeloid differentiation primary response (88) (MyD88) antagonist is a peptide.

17. The device of claim 16 wherein the peptide comprises the amino acid sequence of SEQ ID NO: 22.

18. The device of claim 17 wherein said myeloid differentiation primary response gene (88) (MyD88) antagonist prevents homodimerization of myeloid differentiation primary response (88) (MyD88).

19. The device of claim 15 wherein the nucleic acid is an RNAi agent.

20. The device of claim 19 wherein the RNAi agent is siRNA, shRNA, miRNA, dsRNA or ribozyme or variants thereof.

21. A biomaterial for implantation in or near the spine, wherein said biomaterial is coated or infused with a myeloid differentiation primary response gene (88) (MyD88) antagonist.

22. The biomaterial of claim 21 wherein said biomaterial is for implantation in an intervertebral space.

23. The biomaterial of claim 22 wherein said myeloid differentiation primary response (88) (MyD88) antagonist is a nucleic acid, peptide, protein, antibody or antigen-binding fragment thereof.

24. The biomaterial of claim 23 wherein the myeloid differentiation primary response (88) (MyD88) antagonist is a peptide.

25. The biomaterial of claim 24 wherein the peptide comprises the amino acid sequence of SEQ ID NO: 22.

26. The biomaterial of claim 25 wherein said myeloid differentiation primary response gene (88) (MyD88) antagonist prevents homodimerization of myeloid differentiation primary response (88) (MyD88).

27. The biomaterial of claim 23 wherein the nucleic acid is an RNAi agent.

28. The biomaterial of claim 27 wherein the RNAi agent is siRNA, shRNA, miRNA, dsRNA or ribozyme or variants thereof.

29. A putty for implantation in or near the spine, wherein said putty is coated or infused with a myeloid differentiation primary response gene (88) (MyD88) antagonist.

30. The putty of claim 29 wherein said putty is for implantation in an intervertebral space.

31. The putty of claim 30 wherein said myeloid differentiation primary response (88) (MyD88) antagonist is a nucleic acid, peptide, protein, antibody or antigen-binding fragment thereof.

32. The putty of claim 31 wherein the myeloid differentiation primary response (88) (MyD88) antagonist is a peptide.

33. The putty of claim 32 wherein the peptide comprises the amino acid sequence of SEQ ID NO:22.

34. The putty of claim 33 wherein said myeloid differentiation primary response gene (88) (MyD88) antagonist prevents homodimerization of myeloid differentiation primary response (88) (MyD88).

35. The putty of claim 31 wherein the nucleic acid is an RNAi agent.

36. The putty of claim 35 wherein the RNAi agent is siRNA, shRNA, miRNA, dsRNA or ribozyme or variants thereof.
